(19) United States
(12) Patent Application Publication
(10) Pub. No.: US 2006/0211947 A1
(43) Pub. Date: Sep. 21, 2006

(54) System and method for determining cardiac output

(76) Inventors: Nikolai Krivitski, Ithaca, NY (US); Naveen Thuramalla, Ithaca, NY (US)

Correspondence Address: Randall Reed, 34 Dutch Mill Road, Ithaca, NY, 14850, US

(21) Appl. No.: 11/370,721

(22) Filed: Mar. 7, 2006

Related U.S. Application Data
(60) Provisional application No. 60/659,205, filed on Mar. 7, 2005.

(51) Int. Cl. A61B 5/0275; A61B 5/026

(52) U.S. Cl. 600/504; 600/526

(57) ABSTRACT

A system and method is disclosed for measuring cardiac output. A closed fluid circuit is created between a patient's arterial cannula and venous cannula. A pump regulates flow through the closed fluid circuit. The closed fluid circuit includes at least one port for injection of an indicator. At least one sensor is attached to the closed fluid circuit for taking readings of a blood parameter after the indicator has flowed through the patients system at least once.

### CROSS-REFERENCE TO RELATED APPLICATIONS

The present application claims priority under 35 USC §119 (e) from U.S. provisional application Ser. No. 60/659,205 filed Mar. 7, 2005 and entitled System and Method for Determining Cardiac Output.

### STATEMENT REGARDING FEDERALLY SPONSORED RESEARCH OR DEVELOPMENT

Not applicable.

### REFERENCE TO A “SEQUENCE LISTING”

Not applicable.

### BACKGROUND OF THE INVENTION

1. Field of the Invention

The present invention relates to monitoring the vital signs of a patient and more particularly to monitoring the cardiac output of a patient.

2. Background of the Invention

In providing treatment to a patient in a hospital often the care provider needs to monitor the flow of blood in the patient. Often this includes monitoring the cardiac output, i.e. the amount of blood pumped by the heart. Current systems that use dilution principles require insertion of a catheter into a vein, for example Swan-Ganz thermodilution catheters that require heart catheterization. The catheter is generally about 2 to 4 mm in diameter and can be up to meter in length if not longer. For most adults this is not a problem since their veins and arteries are large and well developed enough to accommodate such a large device. However, for young adolescents and infants it is often impossible to use such inter arterial or venous catheters with sensors due to the small size of their arteries and veins.

There are a number of dilution methods currently available for monitoring cardiac output of children or adolescents that do not require use of catheters with sensors inserted into blood as described above. These methods are: 1) lithium dilution technique and 2) cardio-green dilution technique. The indicator used in the lithium dilution technique exhibits toxicity and requires the physician to carefully monitor the dosage to avoid an overdose. The lithium dilution technique also results in loss of blood to the patient due the need to withdraw blood that is not retuned after it makes contact with the sensor. The cardio-green dilution technique also results in loss of blood to the patient due the need for repetition of administration. Also the repetition of the tests tint the hue of the patient's skin.

Given the problems noted above related to blood loss in small patients that is unacceptable cardiac output in infant is generally monitored or measured by non invasive but less accurate methods such as Color Doppler. However, if an attending physician could safely and with a high degree of accuracy monitor blood flow in infants it would prove a great benefit.

Indicator dilution techniques have been in use for well over a century for determining blood flow including cardiac output. U.S. Pat. No. 6,155,984 describes one method and system by the inventor of the invention described in this specification. In the '984 patent an indicator introduction assembly introduces an indicator into the venous side of the central blood supply. An arterial sensor then determines the concentration of indicator as it passes the sensor after having passed through the left and right side of the heart.

While procedures, as noted above, have been developed that are less invasive than inserting of a 2 to 3 mm in diameter catheter over one meter in length, the systems currently available are still to some extent invasive and specific to certain predefined uses. Thus, what is needed is a simple and effective method and system for measuring cardiac output of an infant or adolescent, that does not required insertion of the catheter into the patient but that allows the measurement procedure to be performed outside of the patient with no blood loss. Additionally, what is needed is a system that is non-invasive, does not contaminate the patient's blood and is easy to implement. Further what is needed is a system that works with existing systems that measure other physical parameters of the patient without interfering with the operation of these existing systems.

### SUMMARY

Thus, it is an objective of the present invention to provide a noninvasive, accurate, and easy to implement method and system for measuring the cardiac output of a patient. It is a further objective to provide a method and system that can be used on infants and adolescents as well as adults. It is an additional objective of the present invention to provide a system that can be easily incorporated into existing patient monitoring and treatment systems.

The present invention achieves these and other objectives by providing: a method for determining cardiac blood output with the steps of: a) establishing an extracorporeal closed fluid circuit between an arterial cannula and a venous cannula pre-existing in an ICU patient; b.) establishing a regulated flow of blood through the closed circuit from the arterial cannula to the venous cannula with a pump; c) injecting an indicator intravenously; d) taking a reading of a blood parameter in the closed fluid circuit after the indicator has flowed at least once through the cardiopulmonary system of the patient to which the arterial and venous cannula are attached; and e) determining cardiac output from the measured blood parameter. In a further aspect of the invention the indicator is injected into the extracorporeal closed fluid circuit.

In another aspect of the present invention it provides a system for determining cardiac output in a patient having: a) a closed fluid extracorporeal circuit connecting an arterial cannula and a venous cannula in a patient; b) a least one sensor on said closed fluid circuit positioned to sense a physical parameter of a fluid flowing in the closed fluid circuit from the arterial cannula to the venous cannula; c) at least one closable access port on the closed fluid circuit, said access port configured to allow fluid access to said closed fluid circuit; d) at least one flow regulator to establish and regulate flow of blood through said closed fluid circuit; e) a processor connected to said sensor configured to receive signals from said at least one sensor and thereby determine cardiac output when blood flows from said arterial cannula to said venous cannula with indicator that has been injected into said at least one closable access port.

### BRIEF DESCRIPTION OF THE DRAWINGS

The invention will be better understood by an examination of the following description, together with the accompanying drawings, in which:

FIG. 1 is a flow chart that shows one method of the system of the present invention;

FIG. 2 is a graph of a time rate of change reading of blood concentration as it might appear during used the method and apparatus;

FIG. 3 is a schematic diagram of one variation of the system of the present invention;

FIG. 3A is a block diagram of the computer/flowmeter combination of FIG. 3;

FIG. 4 is a schematic diagram of another variation of the system of the present invention; and

FIG. 4A is a block diagram of the computer/flowmeter combination of FIG. 4.

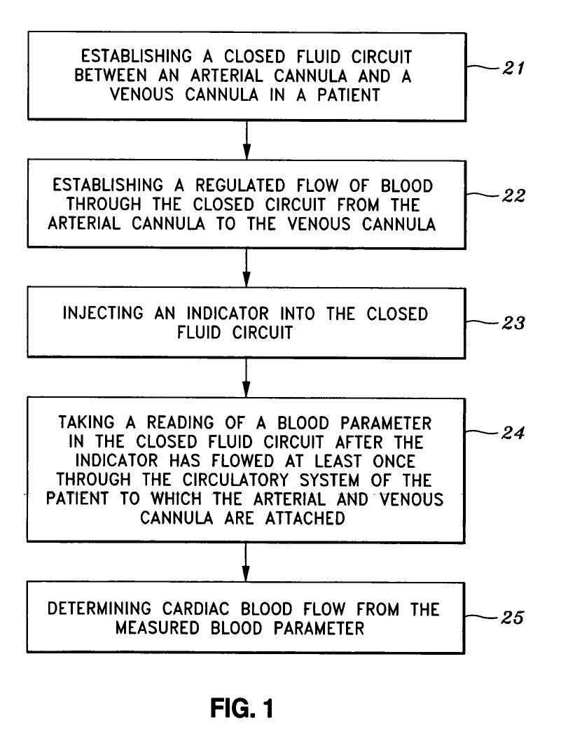

### DETAILED DESCRIPTION OF THE PREFERRED EMBODIMENT

Patients in intensive care unit (ICU) typically have one or more intravenous catheter or cannula and intra-arterial catheter or cannula connected to them. The intravenous cannula or catheter are typically attached to a vein and allow the intravenous feeding of drugs and other substances to the patient. The intra-arterial cannulas or catheters are connected to an artery of the patient and allow the obtaining of direct and precise blood pressure readings. The readings are obtained by connecting pressure cannula to a pressure sensor through the tubing line filled usually with heparinized saline. The saline is slowly delivered into cannula to, prevent clotting.

The actual artery or vein to which the cannula is attached will depend on the requirements of the treatment. Typically the venous cannula is connected to the central venous system which could often be in a jugular vein; or a femoral vein. The arterial cannula is often connected to a radial or a femoral artery or to umbilical artery for neonatals.

A three way or standard valve system is typically connected to the end or to the extension set of the arterial cannula and venous cannula. This allows for easy access to the cannula and ultimately to the vein or artery to which the cannula is attached while allowing the access to be closed and not affect catheter performance. Similarly an intravenous feed (IV) can be inserted into the venous cannula after the appropriate valve is opened. Since it is a three way valve assembly access can be obtained through the third opening to the cannula even though an IV or sensor probe has been inserted into the second opening of the valve.

The present invention utilizes the existing venous and arterial cannula's placed in patients as standard procedure in an ICU. The present invention connects a tube between the arterial cannula and the venous cannula. This tube establishes an extracorporeal closed fluid circuit between the arterial cannula and the venous cannula. Both the arterial and venous cannula have standard valves and access ports openings 49A and 49B which allow for the control of flow of a fluid, i.e. blood flow between each cannula. The standard direction of flow in the tube is from the arterial to the venous cannula. In order to establish an adequate flow rate and avoid clotting of the blood in the extracorporeal closed fluid circuit a pump 53 is added to the line to assist and regulate the flow of blood entering the fluid circuit from the arterial cannula passing through the fluid circuit and exiting at the venous cannula, into the patient.

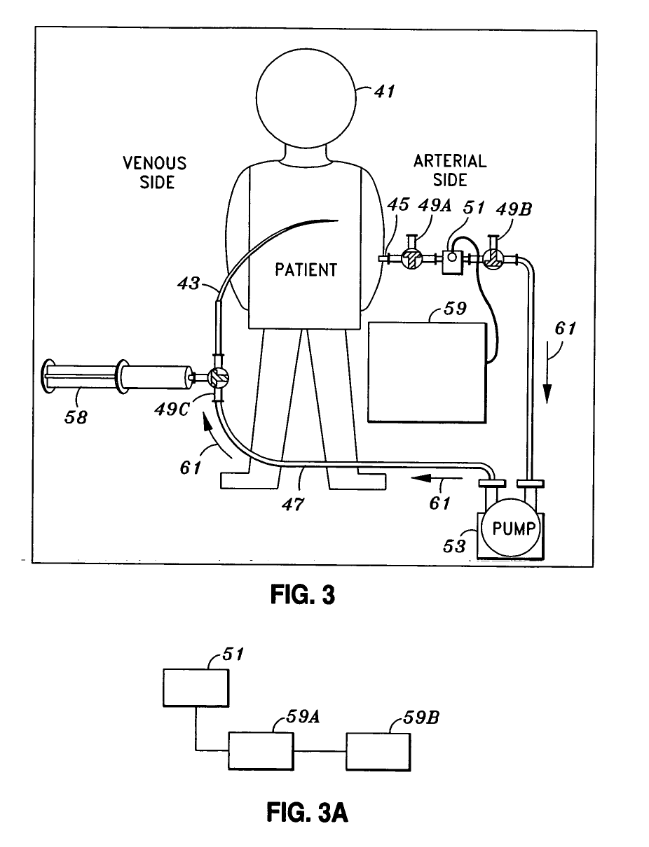

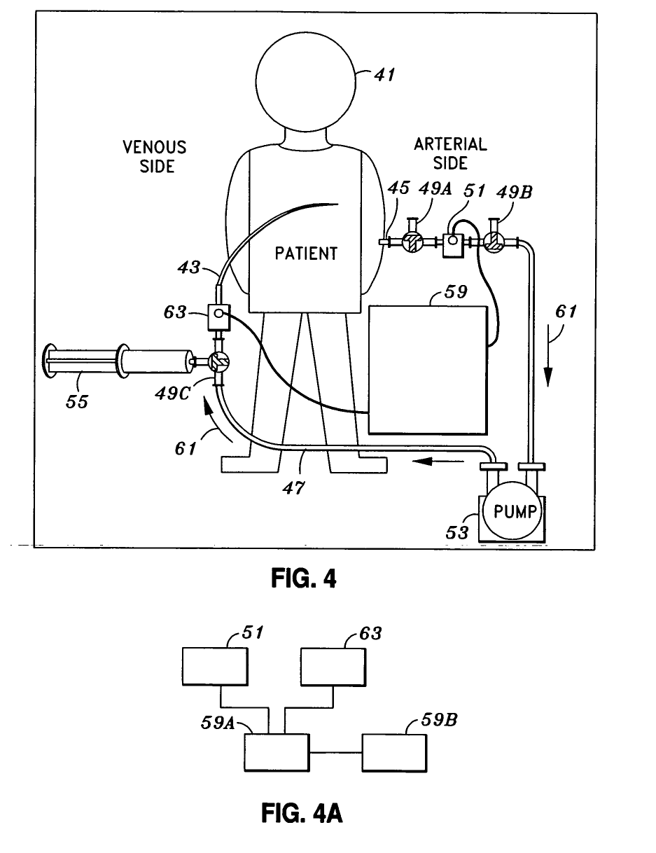

One or two sensors are attached to the fluid circuit to monitor the blood properties and also blood flow if needed in the circuit and take the necessary readings which are used to determine cardiac output. In the preferred embodiment of the invention ultra sound transducers are used. As depicted in FIGS. 3 and 4 the sensor or sensors are connected directly to a combination computer/flowmeter. The computer is running appropriate software to interface with the flowmeter. This software would include the necessary programming the computer would have the necessary hardware to interface with the flow meter which in turn connects to the sensors. FIG. 3A is a block diagram of the computer flowmeter configuration of FIG. 3 with sensor 51 connecting to flow meter 59A which in turn connects to computer 59B. FIG. 4A is a block diagram of the computer flowmeter configuration of FIG. 4 with sensors 51 and 63 connecting to a flowmeter 59A which in turn connects to the computer 59B. In this arrangement meter 59A is a HD02 flowmeter manufactured by Transonic Systems Inc. of Ithaca N.Y., which serves as the interface between the sensors 51 and 63 and computer 59A. The HD02 Flowmeter comes with standard software to interface with the standard personnel computer available from Dell, HP etc. Other configurations of the sensors, meter and computer are possible, such as combining the computer into the flowmeter.

Through the sensors the system monitors cardiac output and based on readings of concentration (or values related, proportional to concentration) of indicator injected into the blood. From readings taken by the system qualitative and quantitative measurements of changes in concentration of indictor are monitored as well as the flow rate in the closed fluid circuit. Additionally, measurement and monitoring of flow through the closed extracorporeal fluid circuit is an added safety factor. Such monitoring can signal problems with flow of blood and clotting in the extracorporeal circuit.

The present invention uses indicator dilution techniques to determine cardiac output. The method of the system in its preferred embodiment includes: establishing an extracorporeal closed fluid circuit between the arterial and venous cannula 21 and starting a flow of blood 22 using a pump in the preferred embodiment, injecting an indicator such as a saline solution, into the closed fluid circuit 23. Blood starts flowing after the valves 49A, 49B and 49C have been opened to allow blood to flow into the extracorporeal closed fluid circuit from the arterial cannula and out through the venous cannula, into the patient. Pump 53 is turned on at this time to create an adequate and regulated flow of blood in the closed fluid circuit. The indicator is injected into the closed fluid circuit after blood has started to flow in the closed fluid circuit. As noted elsewhere indicator can be injected at ports located at 49A, 49B or 49C depending on the purpose of the injection. It should also be noted that in the preferred embodiment when the tubing 47 is connected to the arterial cannula 45 and venous cannula 43 the line 47 has been filled with a saline solution and measurements are taken after blood starts to enter line 47. Additionally, although the preferred embodiment discuss injecting the indicator into line 47 it can be injected intravenously at any point, such as any vein and the invention still practiced.

In the preferred embodiment readings of a blood parameter are taken in the closed fluid circuit after the indicator has flowed at least once through the cardiopulmonary circulatory system of the patient to which the arterial and venous cannula are attached 24. The readings generated by the sensors are collected by the meter and sent to computer where these readings are used to calculate cardiac output 25. The readings taken reflect the concentration of the indicator in the blood passing through the sensor location at the time of the reading. Standard indicator dilution readings are taken and used to calculate the cardiac output. In the preferred embodiment the actual signal sent by the sensor is a change in voltage representative of the speed of ultrasound transmitted through the blood flowing past the sensor location. The variation of the speed of ultrasound in the blood is representative of the concentration of indicator diluted in the blood. Since standard indicator dilution principles are used the following equation is representative of the calculation made:

$$Q = \frac{V}{S}$$

In this equation Q represents the rate of cardiac output, V represents the volume of indicator and S represents the area under the dilution curve. In fact S is the combination of a series of readings of the concentration of indicator take overtime and integrated and thus it is representative of the area under the dilution curve.

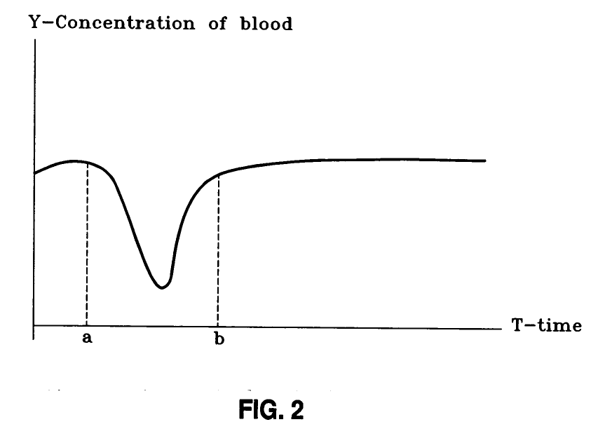

The term S and its definition “area under the dilution curve” are short hand way of referring to the readings taken by the sensor. Another way to view S is as follows, when the sensor detects the presence of indicator, which by this time has passed through the cardiopulmonary circulatory system, it takes a series of readings over time and from these readings calculate S which is graphically represented by FIG. 2. The area from a to b in FIG. 2 is the referred to as the area under the dilution curve. Since in fact the sensor is taking readings of change in concentration over time S can also be represented by the following equation:
S=∫_{b}^{a}B dt
In this equation B is the concentration of blood as it is affected by the presence of indicator in the blood over time. This then is the integral of the time rate of change of concentration of indicator in the blood, the equivalent in integral calculus of the area under the curve. It is understood that in practice the sensor produced voltage changes that are proportional (related) to concentration changes.

In another preferred embodiment of the present invention the standard indicator dilution equation takes the following form:

$$Q = \frac{K \times V}{S}$$

In this equation Q again is the rate of cardiac output, V is the volume of indicator, S is the area under the dilution curve, which is taken from a series of readings over time of the concentration of indicator in the blood flowing past the site of the sensor. K is a calibration constant designed to relate the voltage signal received from the sensor to the concentration of indicator in the blood as it passes the sensor. It is understood that in practice the values derived from the readings are proportional or are related in a quantifiable manner to the concentration of indicator. Calibration is done in the standard manner on site or preferably the sensors, computer software and/or meter are calibrated during manufacture.

In another preferred embodiment of the present invention which involves a two sensor configuaration (FIG. 4) the standard indicator dilution equation takes the following form:

$$Q = \frac{\int ( C ( t ) * V ( t ) ) ⅆ t}{S}$$

In a two sensor configuration of the system (as shown in FIG. 4), one of the sensors is located before the injection site on the venous side while the second one is located on the arterial side. Both sensors can measure the flow through the loop. First sensor records the volume of the injected indicator V(t) and change of concentration C(t) caused due to injection of indicator over the time during which the indicator passes through the sensor site. In this equation Q is the rate of cardiac flow, S is the area under the dilution curve, which is taken form a series of readings over time of the concentration of indicator in the blood flowing past the site of the sensor on the arterial side and ∫(C(t)*V(t))dt represents the amount of indicator injected—which is taken from a series of readings over time of the indicator as it flows past the sensor site on the venous side. The same equation can be used for a one sensor system when the injection is made before the sensor to take an initial calibration reading when the indictor first passes and then record the dilution curve after the indicator has passed through the cardiopulmonary circulation system.

FIG. 3 is a schematic diagram of a single sensor system. An ICU patient 41 has a venous cannula 43 and an arterial cannula 45 attached. An extracorporeal closed fluid circuit 47 connects arterial cannula 45 and venous cannula 43. Closed fluid circuit 47 has one or more access ports 49A 49B and 49C. Sensor 51 connects to closed fluid circuit 47 at the position indicated. Additionally, pump 53, is connected to closed fluid circuit 47. Indicator injection device 55 connects to access port 49C. Sensor 51 connects to flowmeter 59A which connects to computer 59B.

Arterial cannula 45 and venous cannula 43 are typically placed in an ICU patient in a hospital setting. Venous cannula 43 is used to inject into the blood stream of the patient IV's, medications being administered to the patient, as well as other substances that may be necessary for treatment. The injections go directly into the patent's blood system. Typically, the venous cannula is located in the central venous system, which often is a connection to the jugular vein. Other veins such as the femoral vein could be used. The arterial cannula is used to provide access for a catheter to periodically measure blood pressure in the patient and take blood samples. The preferred position for the arterial cannula is typically in the radial artery, or the femoral artery or umbilical artery in case of neonates.

Access valves 49A, 49B, and 49C, are three-way valves, in the preferred embodiment being three-way stopcocks. Three-way valves 49A, 49B, and 49C, allow for the complete stopping of any flow in fluid circuit 47 or allowing the flow of blood through fluid circuit 47 with or without access. Since valves 49A, 49B and 49C are three way valves all three ports of the valve can be opened to allow access to fluid circuit 47 at the same time blood is to flowing through the fluid circuit 47. The type, position and number of these values can be varied without departing from the scope and spirit of the invention.

In the preferred embodiment fluid circuit 47 is typically surgical or medical grade tubing, which extracorporeally connects at one point to arterial cannula 45, and at a second point to venous cannula 43. Extracorporeal closed fluid circuit 47 allows for the flow of blood from arterial cannula 45 to venous cannula 43. In FIG. 3, the standard flow of blood in fluid circuit 47 is from the arterial side, the upstream side, to the venous side, the downstream side. To help regulate the movement of blood through circuit 47, pump 53 is connected. In the preferred embodiment, pump 53 is a peristaltic pump. Peristaltic pump 53 basically massages the tubing of fluid circuit 47 to cause the blood or indicator in the closed fluid circuit to move in the direction of arrows 61. The preferred embodiment uses a peristaltic pump due to its non-invasive nature, the blood remains confined to fluid circuit 47 and does not come into direct contact with the pump. However, many other types of pumps could be used to practice the present invention without departing from the spirit of the present invention.

Indicator injection device 55 in the preferred embodiment is a syringe, which is filled with a saline or other type of indicator; and upon the proper setting of access port valve, 49A, 49B, or 49C, allows for the injection of the indicator into fluid circuit 47.

Sensor 51 in the preferred embodiment is one that measures changes in ultrasound velocity in the blood. The change in velocity being related to the amount of indicator, be it injected by such as in a saline solution or introduced as a temperature change or anyone of a number of different indicators familiar to one of ordinary skill in the art. Sensor 51 connects to flowmeter 59A. In turn Computer 59B receives the readings from flowmeter 59A calculates cardiac output based upon the readings coming from flowmeter 59A, plus additional information which the user of the system inputs into the computer program running on the computer. In the preferred embodiment the program uses the equations set forth above which have been appropriately adapted to perform the calculations necessary to determine cardiac output rate. Sensor 51 clamps around the circuit 47 and thus does not touch and contaminate the blood nor is it contaminated by the blood since they do not touch.

The single sensor system, as depicted in FIG. 3, is used to monitor cardiac output by first connecting the system as depicted in FIG. 3. The next step is commencing of the flow of blood out of the arterial cannula 45 into closed fluid circuit 47 and then back into the patient through venous cannula 43 through a pump 53. Once a flow of blood has been established the indicator is injected by indicator injector 55. Sensor 51 connected to combination computer/flowmeter 59 then begins to monitor the flow of blood through closed fluid circuit 47. When the sensor, 51, has detected indicator previously injected at port 49C it commences its reading. These readings are then captured by flowmeter 59A which then transfers them to computer 59B running the appropriate software. The readings made, in the preferred embodiment as noted above, are with an ultrasound sensor, which includes an ultrasound transducer. These reading, together with the volume of the indicator injected by injector 55 are used to calculate cardiac output. The amount of indicator injected, having been entered by the operator of the system into the software program running on the computer.

The preferred embodiment of the invention actually provides for a calibration reading to translate sensor readings into concentration units. In the preferred embodiment of the single sensor system the calibration reading is obtained by injecting indicator either at port 49A or 49B, to flush the system. The flushing is intended to clear any blood away from sensor 51. Sensor 51 then begins monitoring the flow of fluid initially primed indicator in fluid circuit 47 and upon detecting blood takes a reading to provide a calibration constant. The sensor may factory recalibrated and then these is no need to calibrate it during patient measurements.

FIG. 4 provides a view of a two-sensor configuration of the present invention. In FIG. 4 all of the items that are identical to the items in FIG. 3 have the same numbers. The only substantial addition in FIG. 4 is the addition of a second sensor 63. In the preferred embodiment, the addition of sensor 63 allows for an alternative way to make the initial calibration reading. This eliminates the need to flush the system in order to obtain the calibration reading and allows the taking of the calibration reading based on the one injection of indicator which is used to determine cardiac output. In the variation depicted in FIG. 4 indicator is injected by indicator injector 55 at port 49C. As the indicator initially passes sensor 63 computer/flowmeter 59 takes a calibration reading from sensor 63. When the indicator eventually passes through patient's 41 cardiovascular circulatory systems and out through arterial cannula 45 sensors 51 which is monitoring flow in fluid circuit 47 takes the necessary readings from which cardiac output is calculated.

Although a saline indicator is used in the preferred embodiment any suitable indicator can be used. Other types of indicators could include a change in one or more blood characteristics, etc. These include but are not limited to thermo dilution.

The preferred embodiment described above uses a sudden injection of indicator, i.e. a bolus, to obtain the necessary reading. However, it should be noted that the present invention could be practiced with variations of the indicator dilution technique and not depart from the spirit of the present invention. Thus, the invention could also be practiced with a steady state indicator dilution technique.

The current invention is described using a preferred embodiment that uses indicator dilution, such as injecting a saline solution and sensing the presence of indicator and its concentration with an ultrasound sensor. Those of ordinary skill in the art, once they understand the principles of the current invention will realize that other physical properties of the blood can be changed and other types of sensors can be used to obtain the dilution curves without departing from the spirit of the invention. Among potential changes in blood properties possible are its optical properties, electrical proprieties (electrical impedance), thermal properties, or any other appropriate physical or chemical properties of the blood. Accordingly, optical sensors, electrical sensors, thermal sensors, or other appropriate physical or chemical sensors can be used maybe used depending on the change in the property of the blood made. Additionally, isotope tracers with appropriate sensors could be used. This is not meant to be an exhaustive list. The equations can be modified to work with different indicators which will not depart form the spirit of the invention. Also, the

While the invention has been particularly shown and described with reference to a preferred embodiment thereof, it will be understood by those skilled in the art that various changes in form and detail may be made to it without departing from the spirit and scope of the invention.

I claim:

1. A method for determining cardiac output comprising the steps of:
a) establishing an extracorporeal closed fluid circuit between an arterial cannula and a venous cannula in an ICU patient;
b.) establishing a regulated flow of blood through the closed circuit from the arterial cannula to the venous cannula, using a pump;
c) injecting an indicator intravenously;
d) taking a reading of a blood parameter in the closed fluid circuit after the indicator has flowed at least once through the cardiopulmonary circulatory system of the patient to which the arterial and venous cannula are attached; and
e) determining cardiac output from the measured blood parameter.

2. The method of claim 1 comprising the additional step of taking a calibration reading in addition to taking the reading of the blood parameter to thereby improve the accuracy of determining cardiac output.

3. The method of claim 1 wherein the step of establishing the regulated blood flow comprises the step of starting a pump to cause the blood to flow at a predetermined rate.

4. The method of claim 1 wherein the step of taking a reading comprises monitoring the flow of blood in the closed circuit and taking a series of readings overtime when there is an indication of the presence of indicator in the blood.

5. The method of claim 4 wherein the step of determining includes the step of calculating blood flow using the equation:
$$Q = \frac{V}{S}$$
wherein Q represents cardiac blood flow, V represent the amount of indicator injected and S is representative of the integral of the time rate of change in concentration of blood to indicator injected when the indicator is detected as present in the blood.

6. The method of claim 5 wherein S, the time rate of change of concentration of blood to indicator, is represented as an area under a dilution curve.

7. The method of claim 4 comprising the additional step of taking a calibration reading in addition to taking the reading of the blood parameter to thereby improve the accuracy of determining cardiac blood flow.

8. The method of claim 7 wherein the step of determining includes the step of calculating blood flow using the equation:
$$Q = \frac{K \times V}{S}$$
wherein Q represents cardiac blood flow, V represent the amount of indicator injected, K is a constant obtained from taking the calibration reading and S is representative of the integral of the time rate of change in concentration of blood to indicator injected when the indicator is detected as present in the blood.

9. The method of claim 1 wherein the reading is taken with a single sensor.

10. The method of claim 9 comprising the additional step of taking a calibration reading in addition to taking the reading of the blood parameter to thereby improve the accuracy of determining cardiac blood output.

11. The method of claim 10 including the further step of flushing the closed fluid circuit prior to taking the calibration reading.

12. The method of claim 11 wherein the step of taking the reading of a blood parameter and the calibration reading includes the step of positioning the sensor adjacent to and downstream from the arterial cannula.

13. The method of claim 1 including the further step of positioning a first sensor and a second sensor along the closed fluid loop.

14. The method of claim 13 comprising the additional step of taking a calibration reading in addition to taking the reading of the blood parameter to thereby improve the accuracy of determining cardiac blood output.

15. The method of claim 14 wherein the step taking the calibration reading comprises taking the calibration reading with the first sensor and the step of taking the reading of the blood parameter, taking it with the second sensor.

16. The method of claim 15 wherein the step of positioning a first sensor and a second sensor along the closed fluid loop, comprises positioning the first sensor adjacent to and upstream from the venous cannula and the second sensor adjacent to and downstream from the arterial cannula.

17. The method of claim 16 wherein the step of injecting an indicator into the closed fluid circuit comprises injecting it into the closed fluid circuit at a point upstream from the first sensor and downstream from the second sensor.

18. The method of claim 1 wherein the step of establishing a closed fluid circuit comprises connecting a first end of a flexible tube to the arterial cannula and second end of the flexible tube to the venous cannula.

19. The method of claim 1 wherein the step of taking a reading is taking a reading with an ultrasonic transducer.

20. The method of claim 1 wherein the step or injecting an indicator includes injecting a saline solution.

21. The method of claim 1 wherein the step of injecting an indicator intravenously is injecting it into the closed fluid circuit.

22. A system for determining cardiac output in a patient comprising:
a) a closed fluid circuit connecting an arterial cannula and a venous cannula in a patient;
b) a least one sensor on said closed fluid circuit positioned to sense a physical parameter of a fluid flowing in the closed fluid circuit from the arterial cannula to the venous cannula;
c) at least one closable access port on the closed fluid circuit, said access port configured to allow fluid access to said closed fluid circuit;
d) at least one flow regulator to establish and regulate flow of blood through said closed fluid circuit;
e) a processor connected to said sensor configured to receive signals from said at least one sensor and thereby determines cardiac output when blood flows from said arterial cannula to said venous cannula with indicator that has been injected into said at least one closable access port.

23. The system of claim 21 wherein said flow regulator is a pump.

24. The system of claim 22 wherein said pump is peristaltic pump.
